(19) United States
(12) Patent Application Publication
(10) Pub. No.: US 2020/0121230 A1
(43) Pub. Date: Apr. 23, 2020

(54) APPARATUS AND METHOD FOR ESTIMATING ANALYTE CONCENTRATION

(71) Applicant: SAMSUNG ELECTRONICS CO., LTD., Suwon-si (KR)

(72) Inventor: June Young LEE, Seongnam-si (KR)

(73) Assignee: SAMSUNG ELECTRONICS CO., LTD., Suwon-si (KR)

(21) Appl. No.: 16/654,729

(22) Filed: Oct. 16, 2019

(30) Foreign Application Priority Data
Oct. 23, 2018 (KR) 10-2018-0126622

(51) Int. Cl. A61B 5/145; A61B 5/1455; A61B 5/00; G16H 10/40; G06F 1/16; G16H 40/63

(52) U.S. Cl. CPC A61B 5/14532; A61B 5/14546; A61B 5/1455; G16H 40/63; G16H 10/40; G06F 1/163; A61B 5/4845

(57) ABSTRACT

An apparatus for estimating an analyte concentration includes a spectrum acquisition device configured to obtain a plurality of in vivo spectra for training which are measured during a first interval, and obtain an in vivo spectrum for analyte concentration estimation which is measured during a second interval, and a processor configured to generate a plurality of candidate concentration estimation models by varying a number of principal components based on the plurality of in vivo spectra for training, obtain a plurality of residual vectors corresponding to the plurality of in vivo spectra for training by using the plurality of candidate concentration estimation models, select a candidate concentration estimation model, from among the plurality of candidate concentration estimation models, based on the plurality of residual vectors, and estimate the analyte concentration by using the selected candidate concentration estimation model and the in vivo spectrum for analyte concentration estimation.

### CROSS-REFERENCE TO RELATED APPLICATION(S)

This application is based on and claims priority under 35 U.S.C. § 119 to Korean Patent Application No. 10-2018-0126622, filed on Oct. 23, 2018, in the Korean Intellectual Property Office, the disclosure of which is incorporated by reference herein in its entirety.

### BACKGROUND

### 1. Field

The following description relates to an apparatus and method for estimating the concentration of an in vivo analyte from a bio-signal.

### 2. Description of Related Art

Diabetes is a chronic and incurable disease that causes various complications, such that people with diabetes are advised to check their blood glucose regularly to prevent complications. In particular, when insulin is administered to control blood glucose levels, the blood glucose levels should be closely monitored to avoid hypoglycemia and control insulin dosage. An invasive method of finger pricking is generally used to measure blood glucose levels. However, while the invasive method may provide high reliability in measurement, it may cause pain and inconvenience as well as an increased risk of infections due to the use of injection. Recently, research has been conducted on a method of non-invasively measuring blood glucose levels by using a spectrometer without blood sampling.

### SUMMARY

Provided is an apparatus and method for estimating the concentration of an in vivo analyte using a bio-signal.

In accordance with an aspect of the disclosure, provided is an apparatus for estimating an analyte concentration includes a spectrum acquisition device configured to obtain a plurality of in vivo spectra for training which are measured during a first interval, and obtain an in vivo spectrum for analyte concentration estimation which is measured during a second interval, and a processor configured to generate a plurality of candidate concentration estimation models by varying a number of principal components based on the plurality of in vivo spectra for training, obtain a plurality of residual vectors corresponding to the plurality of in vivo spectra for training by using the plurality of candidate concentration estimation models, select a candidate concentration estimation model, from among the plurality of candidate concentration estimation models, based on the plurality of residual vectors, and estimate the analyte concentration by using the selected candidate concentration estimation model and the in vivo spectrum for analyte concentration estimation.

The processor may generate the plurality of candidate concentration estimation models using a Net Analyte Signal (NAS) algorithm.

The plurality of residual vectors may represent differences between generated in vivo spectra, generated using the plurality of concentration estimation models, and actually measured in vivo spectra.

The processor may extract a predetermined number of principal component vectors by analyzing the plurality of in vivo spectra for training, based on varying the number of principal components, obtain a plurality of inverse matrices of matrices composed of the varied number of principal component vectors and a pure component spectrum vector of an analyte, generate a plurality of candidate concentration estimation model matrices based on the plurality of inverse matrices, and generate the plurality of candidate concentration estimation models based on the plurality of candidate concentration estimation model matrices.

The processor may extract the predetermined number of principal component vectors by using one of Principal Component Analysis (PCA), Independent Component Analysis (ICA), Non-negative Matrix Factorization (NMF), and Singular Value Decomposition (SVD).

The processor may extract a plurality of component vectors, corresponding to the analyte, from the plurality of candidate concentration estimation model matrices, determine angles between the plurality of extracted component vectors and the plurality of residual vectors, determine a number of principal components, at which a value obtained by multiplying a magnitude of a residual vector, of the plurality of residual vectors, by an absolute value of cosine of the angle is maximum, and select the candidate concentration estimation model, generated by using the determined number of principal components, from among the plurality of generated candidate concentration estimation models.

The spectrum acquisition device may receive the plurality of in vivo spectra for training and the in vivo spectrum for analyte concentration estimation from an external device.

The spectrum acquisition device may measure the plurality of in vivo spectra for training and the in vivo spectrum for analyte concentration estimation by emitting light towards an object and receiving light reflected by or scattered from the object.

The first interval may be an interval in which the analyte concentration of is substantially constant.

The analyte may be at least one of glucose, triglycerides, urea, uric acid, lactate, proteins, cholesterol, or ethanol.

The analyte may be glucose, and the first interval may be a fasting interval.

In accordance with an aspect of the disclosure, a method of estimating an analyte concentration may include obtaining a plurality of in vivo spectra for training which are measured during a predetermined interval, generating a plurality of candidate concentration estimation models by varying a number of principal components based on the plurality of in vivo spectra for training, obtaining a plurality of residual vectors corresponding to the plurality of in vivo spectra for training by using the plurality of candidate concentration estimation models, selecting a candidate concentration estimation model, from among the plurality of candidate concentration estimation models, based on the plurality of residual vectors, and estimating the analyte concentration by using the selected concentration estimation model.

The generating of the plurality of candidate concentration estimation models by varying the number of principal components may include generating the plurality of candidate concentration estimation models using a Net Analyte Signal (NAS) algorithm.

The plurality of residual vectors may represent differences between a plurality of generated in vivo spectrum, generating using the plurality of concentration estimation models, and a plurality of actually measured in vivo spectra.

The generating of the plurality of candidate concentration estimation models by varying the number of principal components may include extracting a predetermined number of principal component vectors by analyzing the plurality of in vivo spectra for training, based on varying the number of principal components, obtaining a plurality of inverse matrices of matrices composed of the varied number of principal component vectors and a pure component spectrum vector of an analyte, generating a plurality of candidate concentration estimation model matrices based on the plurality of inverse matrices, and generating the plurality of candidate concentration estimation models based on the plurality of candidate concentration estimation model matrices.

The extracting of the predetermined number of principal component vectors may include extracting the predetermined number of principal component vectors by using one of Principal Component Analysis (PCA), Independent Component Analysis (ICA), Non-negative Matrix Factorization (NMF), and Singular Value Decomposition (SVD).

The selecting of the candidate concentration estimation model may include extracting a plurality of component vectors, corresponding to the analyte, from the plurality of candidate concentration estimation model matrices, determining angles between the plurality of component vectors and the plurality of residual vectors, determining a number of principal components, at which a value obtained by multiplying a magnitude of a residual vector, of the plurality of residual vectors, by an absolute value of cosine of the angle is maximum, and selecting the candidate concentration estimation model, generated by using the determined number of principal components, from among the plurality of candidate concentration estimation models.

The predetermined interval may be an interval in which the analyte concentration is substantially constant.

The analyte may be at least one of glucose, triglycerides, urea, uric acid, lactate, proteins, cholesterol, or ethanol.

The analyte may be glucose, and the predetermined interval may be a fasting interval.

### BRIEF DESCRIPTION OF THE DRAWINGS

The above and other aspects, features, and advantages of certain embodiments of the present disclosure will be more apparent from the following description taken in conjunction with the accompanying drawings, in which:

FIGS. 1 and 2 are diagrams explaining a concept of a Net Analyte Signal (NAS) algorithm according to an embodiment.

FIG. 3 is a block diagram illustrating an example of an apparatus for estimating a concentration according to an embodiment.

FIGS. 4A through 4J are diagrams explaining a correlation between a value of Norm(Residual)*abs(cos θ) according to a number of principal components and an estimation result of blood glucose using a concentration estimation model generated based on the number of principal components according to an embodiment.

FIG. 5 is a block diagram illustrating an apparatus for estimating a concentration according to an embodiment.

FIG. 6 is a flowchart illustrating a method of estimating a concentration according to an embodiment.

FIG. 7 is a block diagram illustrating a system for estimating a concentration according to an embodiment.

FIG. 8 is a diagram illustrating an example of a wrist-type wearable device according to an embodiment.

Throughout the drawings and the detailed description, unless otherwise described, the same drawing reference numerals may refer to the same elements, features, and structures. The relative size and depiction of these elements may be exaggerated for clarity, illustration, and convenience.

### DETAILED DESCRIPTION

Hereinafter, embodiments of the present disclosure will be described in detail with reference to the accompanying drawings. It should be noted that, wherever possible, the same reference symbols may refer to the same parts even in different drawings. In the following description, a detailed description of known functions and configurations incorporated herein may be omitted so as to not obscure the subject matter of the present disclosure.

Process steps described herein may be performed differently from a specified order, unless a specified order is clearly stated in the context of the disclosure. That is, each step may be performed in a specified order, in a different order, at substantially the same time, or in a reverse order.

Further, the terms used throughout this specification may be defined in consideration of the functions according to embodiments, and can be varied according to a purpose of a user or manager, precedent, and or the like. Therefore, definitions of the terms may be made on the basis of the overall context of the disclosure.

It should be understood that, although the terms “first,” “second,” etc. may be used herein to describe various elements, these elements might not be limited by these terms. These terms may be used to distinguish one element from another. Any references to a singular term may include a plural form of the term unless expressly stated otherwise. In the present specification, it should be understood that the terms, such as “including,” “having,” etc., are intended to indicate the existence of the features, numbers, steps, actions, components, parts, or combinations thereof disclosed in the specification, and are not intended to preclude the possibility that one or more other features, numbers, steps, actions, components, parts, or combinations thereof may exist or may be added.

Further, components that will be described in the specification might be discriminated according to functions mainly performed by the components. That is, two or more components may be integrated into a single component. Furthermore, a single component may be separated into two or more components. Moreover, each component may additionally perform some or all of a function executed by another component in addition to the main function thereof. Some or all of the main function of each component which will be explained may be carried out by another component.

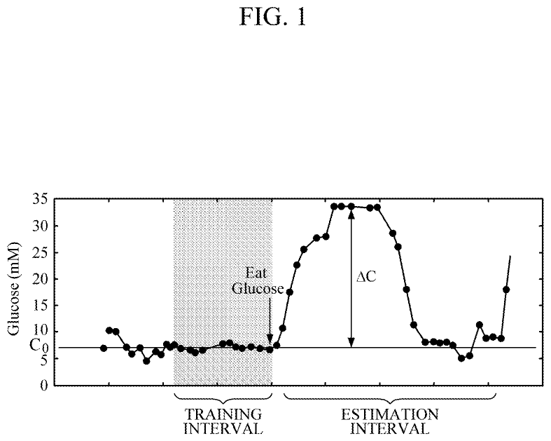

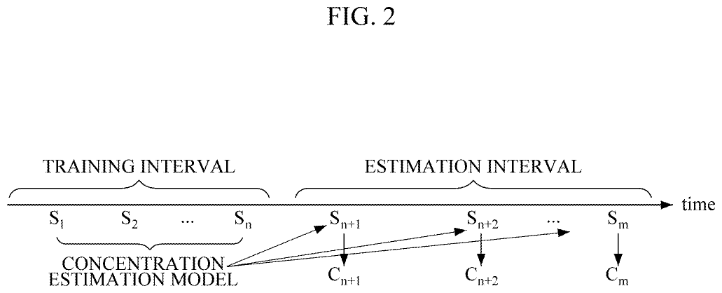

FIGS. 1 and 2 are diagrams explaining a concept of a Net Analyte Signal (NAS) algorithm according to an embodiment.

Referring to FIGS. 1 and 2, the Net Analyte Signal (NAS) algorithm may generate an analyte concentration estimation model by identifying a spectrum change factor, which is relatively irrelevant to a change in an analyte concentration, using in vivo spectra S₁, S₂, . . . , and Sₙ measured during a training interval (e.g., timeframe) as training data. Further, the NAS algorithm may estimate analyte concentrations Cₙ₊₁, Cₙ₊₂ . . . , and Cₘ by using in vivo spectra Sₙ₊₁, Sₙ₊₂, . . . , and Sₘ measured during an estimation interval following the training interval and the generated concentration estimation model. In this case, the training interval may be an interval (e.g., a fasting interval if an analyte is glucose) in which the concentration of an in vivo analyte is substantially constant. As used herein, a concentration of an in vivo analyte being “substantially constant” may refer to a change in the concentration of the in vivo analyte being less than a predetermined threshold. As an example, and referring to FIG. 1, the glucose concentration may be substantially constant in the training interval because a change in the concentration is not greater than substantially five millimolar (mM). It should be understood that a threshold change value for “substantially constant” may vary depending on the underlying value that remains “substantially constant.”

That is, the NAS algorithm may generate a concentration estimation model based on the in vivo spectra measured during the training interval, and then may estimate an analyte concentration by applying the generated concentration estimation model to the in vivo spectra measured during the estimation interval.

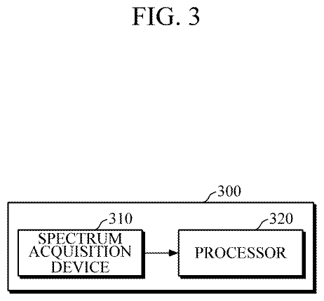

FIG. 3 is a block diagram illustrating an apparatus for estimating a concentration according to an embodiment. The concentration estimating apparatus 300 of FIG. 3 is an apparatus for estimating an analyte concentration by analyzing an in vivo spectrum of an object, and may be embedded in an electronic device. Further, the concentration estimating apparatus 300 of FIG. 3 may be enclosed in a housing to be provided as a separate device. In this case, examples of the electronic device may include a cellular phone, a smartphone, a tablet personal computer (PC, a laptop computer, a personal digital assistant (PDA), a portable multimedia player (PMP), a navigation device, an MP3 player, a digital camera, a wearable device, and the like; and examples of the wearable device may include a wristwatch-type wearable device, a wristband-type wearable device, a ring-type wearable device, a waist belt-type wearable device, a necklace-type wearable device, an ankle band-type wearable device, a thigh band-type wearable device, a forearm band-type wearable device, and the like. However, the electronic device is not limited to the above examples, and the wearable device is neither limited thereto.

Referring to FIG. 3, the concentration estimating apparatus 300 includes a spectrum acquisition device 310 and a processor 320.

The spectrum acquisition device 310 may obtain an in vivo spectrum of an object. For example, the spectrum acquisition device 310 may obtain an in vivo spectrum measured during an interval in which an analyte concentration of an object is substantially constant (hereinafter referred to as an “in vivo spectrum for training”) and/or an in vivo spectrum measured for estimating an analyte concentration of an object (hereinafter referred to as an “in vivo spectrum for estimation”).

In an embodiment, the spectrum acquisition device 310 may obtain an in vivo spectrum by receiving the in vivo spectrum from an external device which measures and/or stores in vivo spectra. In this case, the spectrum acquisition device 310 may use various communication techniques such as Bluetooth communication, Bluetooth Low Energy (BLE) communication, Near Field Communication (NFC), WLAN communication, Zigbee communication, Infrared Data Association (IrDA) communication, wireless fidelity (Wi-Fi) communication, Ultra-Wideband (UWB) communication, Ant+ communication, Wi-Fi Direct (WFD) communication, Radio Frequency Identification (RFID) communication, third generation (3G) communication, fourth generation (4G) communication, fifth generation (5G) communication, and the like.

In an embodiment, the spectrum acquisition device 310 may obtain an in vivo spectrum by directly measuring an in vivo spectrum by emitting light towards an object and receiving light reflected by or scattered from the object. In this case, the spectrum acquisition device 310 may measure the in vivo spectrum by using Infrared spectroscopy, Raman spectroscopy, or the like, and may also use various spectroscopic methods. To this end, the spectrum acquisition device 310 may include a light source which emits light towards an object, and a photodetector which measures an in vivo spectrum by receiving light reflected by or scattered from the object.

The light source may emit near infrared rays (NIR) or mid infrared rays (MIR). However, wavelengths of light to be emitted by the light source may vary according to a purpose of measurement or the types of an analyte. Further, the light source may be a single light-emitting body, or may be formed as an array of a plurality of light-emitting bodies. The light source may include a light emitting diode (LED), a laser diode, a fluorescent body, and the like.

The photodetector may include a photo diode, a photo transistor (PTr), a charge-coupled device (CCD), and the like. The photodetector may be a single device, or may be formed as an array of a plurality of devices.

There may be various numbers and arrangements of light sources and photodetectors, and the number and arrangement thereof may vary according to the types and a purpose of use of an analyte, the size and shape of the electronic device in which the concentration estimating apparatus 300 is embedded, and the like.

The processor 320 may control the overall operation of the concentration estimating apparatus 300.

According to predetermined intervals or at a user's request, the processor 320 may control the spectrum acquisition device 310 to obtain the in vivo spectrum for training and/or the in vivo spectrum for estimation.

Based on the spectrum acquisition device 310 obtaining a plurality of in vivo spectra for training, the processor 320 may generate a plurality of candidate concentration estimation models based on the plurality of obtained in vivo spectra for training, and may select a concentration estimation model from among the generated plurality of candidate concentration estimation models. In an embodiment, the processor 320 may generate the plurality of candidate concentration estimation models based on the NAS algorithm by using the plurality of in vivo spectra for training. In this case, examples of the analyte may include glucose, triglycerides, urea, uric acid, lactate, proteins, cholesterol, ethanol, and the like, but the analyte is not limited thereto. In the case where an in vivo analyte is glucose, an analyte concentration may indicate a blood glucose level; and an interval in which an analyte is substantially constant may indicate a fasting interval in which glucose is consumed by an object. Hereinafter, for convenience of explanation, the following description will be made using glucose as an example of an analyte.

The processor 320 may extract a predetermined number of principal component vectors by analyzing the plurality of in vivo spectra for training. For example, the processor 320 may extract a predetermined number of principal component vectors from the plurality of in vivo spectra for training, which are measured during the fasting interval, by using various dimension reduction algorithms such as Principal Component Analysis (PCA). Independent Component Analysis (ICA). Non-negative Matrix Factorization (NMF), Singular Value Decomposition (SVD), and the like.

The processor 320 may generate a plurality of candidate concentration estimation models by varying the number of principal components based on the predetermined number of extracted principal component vectors. For example, upon extracting five principal component vectors PC1 to PC5 by analyzing the plurality of in vivo spectra for training, the processor 320 may generate a candidate concentration estimation model M1 by using one principal component vector PC1; may generate a candidate concentration estimation model M2 by using two principal component vectors PC1 and PC2; may generate a candidate concentration estimation model M3 by using three principal component vectors PC1 to PC3; may generate a candidate concentration estimation model M4 by using four principal component vectors PC1 to PC4; and may generate a candidate concentration estimation model M5 by using five principal component vectors PC1 to PC5. In this case, the generated candidate concentration estimation models may be represented by the following Equation 1.

$$[ \begin{matrix}{C}_{1} \\ {C}_{2} \\ ⋮ \\ {C}_{k} \\ {C}_{g}\end{matrix} ] = {[ \begin{matrix}{PC}_{1} \\ {PC}_{2} \\ ⋮ \\ {PC}_{k} \\ {ɛ}_{g}\end{matrix} ]}^{- 1} * {S}_{skin} / L = [ \begin{matrix}{PC}_{1 -} NAS \\ {PC}_{2 -} NAS \\ ⋮ \\ {PC}_{k -} NAS \\ {glucose}_{k -} NAS\end{matrix} ] * {S}_{skin} / L \qquad [ Equation 1 ]$$

Herein, C₁, C₂, and Cₖ denote concentrations of principal components; C_{g} denotes an analyte concentration; PC₁, PC₂, and PCₖ denote principal component vectors; ε_{g} denotes a spectrum of an analyte per unit concentration (e.g., 1 mM) (hereinafter referred to as a pure component spectrum); Sₛₖᵢₙ denotes an in vivo spectrum vector; L represents an optical path length; and k denotes the number of principal components. Further, PC₁_NAS and PCₖ_NAS denote component vectors of a candidate concentration estimation model matrix corresponding to principal components; glucoseₖ_NAS denotes a component vector of a concentration estimation model matrix corresponding to an analyte; and ε_{g} may be obtained experimentally.

That is, upon varying the number of principal components, the processor 320 may obtain an inverse matrix of a matrix composed of the varied number of principal component vectors and the pure component spectrum vector of an analyte, to generate a plurality of candidate concentration estimation model matrices; and may generate a plurality of candidate concentration estimation models based on the plurality of generated candidate concentration estimation model matrices.

The processor 320 may obtain a residual vector for each of the plurality of in vivo spectra for training, by using the plurality of candidate concentration estimation models generated by varying the number of principal components. In this case, the residual vector may represent a difference between an in vivo spectrum, reconstructed using the concentration estimation model, and an actually measured in vivo spectrum. For example, the processor 320 may determine principal component concentrations C_{n,1}, C_{n,2}, and C_{n,k}, and an analyte concentration C_{n,g} for each of the in vivo spectra for training by using Equation 1, and may reconstruct each of the in vivo spectra for training by using Equation 2 shown below, to generate a vector Sᵣₑ_{_{n,k}} of the reconstructed in vivo spectrum for training. In addition, the processor 320 may obtain a residual vector Residual_{n,k} of each of the in vivo spectra for training by using Equation 3 shown below.

$$\begin{matrix}{S}_{{re}_{n , k}} = [ \begin{matrix}{PC}_{1} \\ {PC}_{2} \\ ⋮ \\ {PC}_{k} \\ {ɛ}_{g}\end{matrix} ] * [ \begin{matrix}{C}_{n , 1} \\ {C}_{n , 2} \\ ⋮ \\ {C}_{n , k} \\ {C}_{n , g}\end{matrix} ] * L & [ Equation 2 ] \\ {Residual}_{n , k} = {S}_{training \_ {skin}_{n} - {S}_{{re}_{n , k}}} & [ Equation 3 ]\end{matrix}$$

Herein, n denotes an index of the in vivo spectrum for training; and S_{training_skin}_{ₙ} denotes a vector of the in vivo spectrum for training.

The processor 320 may extract a component vector glucoseₖ_NAS corresponding to an analyte, from the plurality of candidate concentration estimation model matrices generated by varying the number of principal components; and may select one of the plurality of candidate concentration estimation models based on the extracted component vector glucoseₖ_NAS, corresponding to the analyte, and the residual vector Residual_{n,k}.

In an embodiment, the processor 320 may determine an angle θ_{n,k} between the component vector glucoseₖ_NAS corresponding to the analyte and the residual vector Residual_{n,k}, and may determine the number k of principal components, at which a value obtained by multiplying a magnitude of the residual vector Residual_{n,k} by an absolute value of cos θ_{n,k} is maximum. Further, among the plurality of candidate concentration estimation models generated for each number of principal components, the processor 320 may select, as an optimal concentration estimation model, a candidate concentration estimation model generated using the number k of principal components, at which a value of Norm(Residual_{n,k})*abs(cos θ_{n,k}) obtained by multiplying a magnitude of the residual vector Residual_{n,k} by an absolute value of cos θ_{n,k} is maximum.

Upon selecting the optimal concentration estimation model, and then obtaining an in vivo spectrum for estimation, which is used for estimating an analyte concentration, the processor 320 may estimate an analyte concentration by using the obtained in vivo spectrum for estimation and the selected concentration estimation model. For example, the processor 320 may estimate the analyte concentration by using Equation 1 shown elsewhere herein.

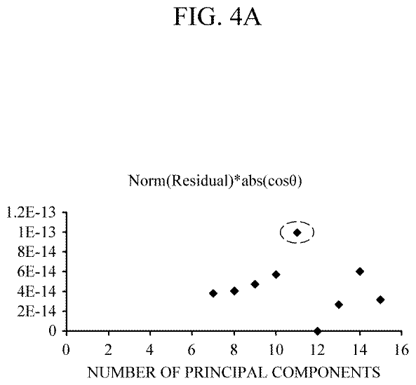

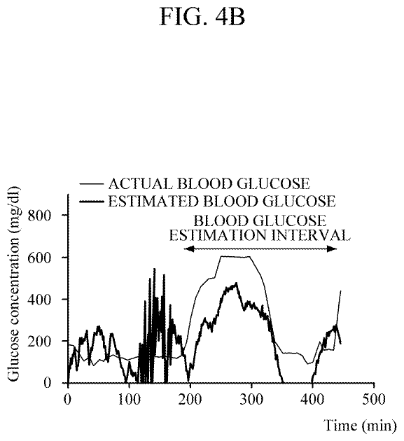

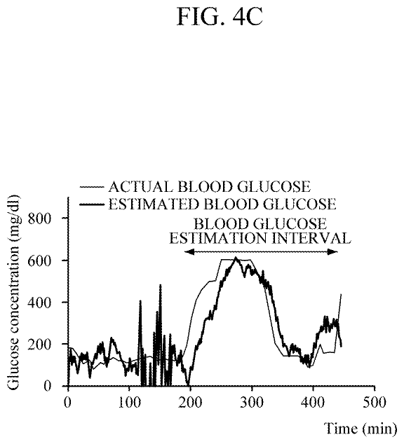

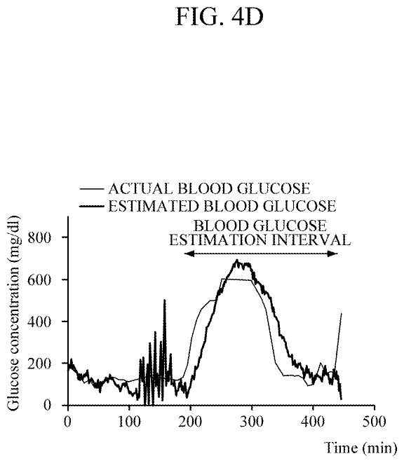

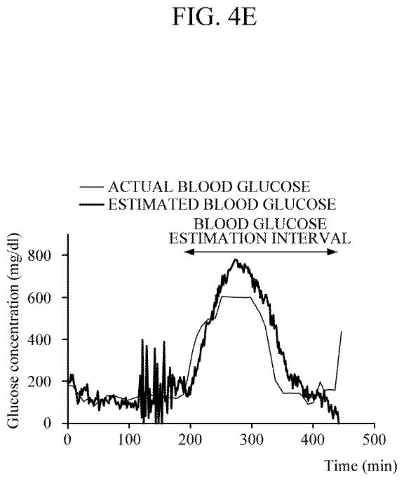

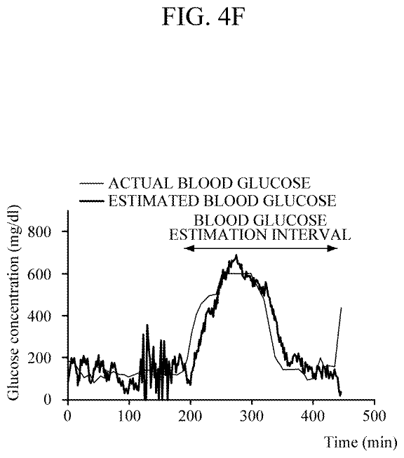

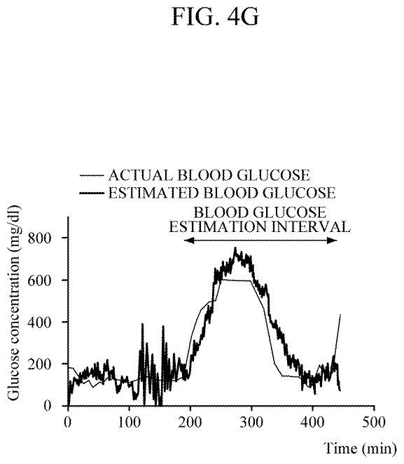

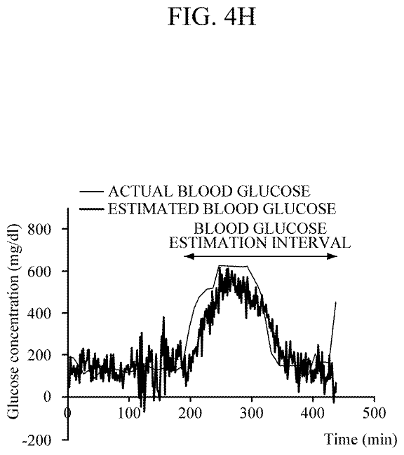

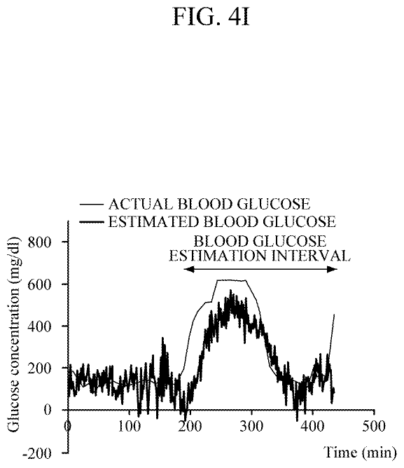

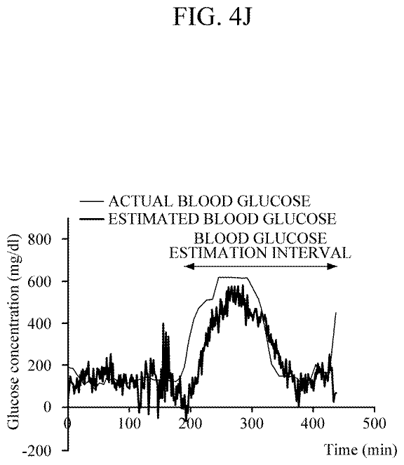

FIGS. 4A through 4J are exemplary diagrams explaining a correlation between the value of Norm(Residual)*abs(cos θ) based on the number of principal components and an estimation result of blood glucose using the concentration estimation model generated based on the number of principal components. FIG. 4A illustrates a diagram illustrating an example of the value of Norm(Residual)*abs(cos θ) based on the number of principal components; FIG. 4B illustrates a diagram illustrating an example of an estimation result of blood glucose based on a concentration estimation model generated using 7 principal components; FIG. 4C illustrates a diagram illustrating an example of an estimation result of blood glucose based on a concentration estimation model generated using 8 principal components; FIG. 4D illustrates a diagram illustrating an estimation result of blood glucose based on a concentration estimation model generated using 9 principal components; FIG. 4E illustrates a diagram illustrating an estimation result of blood glucose based on a concentration estimation model generated using 10 principal components; FIG. 4F illustrates a diagram illustrating an estimation result of blood glucose based on a concentration estimation model generated using 11 principal components; FIG. 4G illustrates a diagram illustrating an estimation result of blood glucose based on a concentration estimation model generated using 12 principal components; FIG. 4H illustrates a diagram illustrating an estimation result of blood glucose based on a concentration estimation model generated using 13 principal components; FIG. 4I illustrates a diagram illustrating an estimation result of blood glucose based on a concentration estimation model generated using 14 principal components; and FIG. 4J illustrates a diagram illustrating an estimation result of blood glucose based on a concentration estimation model generated using 15 principal components.

Referring to FIGS. 4A through 4J, it can be seen that when the number of principal components is 11, the value of Norm(Residual)*abs(cos θ) is maximum, at which accuracy of an estimated blood glucose value is greatest. That is, by selecting the number of principal components, at which the value of Norm(Residual)*abs(cos θ) is maximum, and by estimating blood glucose based on the concentration estimation model generated using the selected number of principal components, accuracy in estimating blood glucose may be improved.

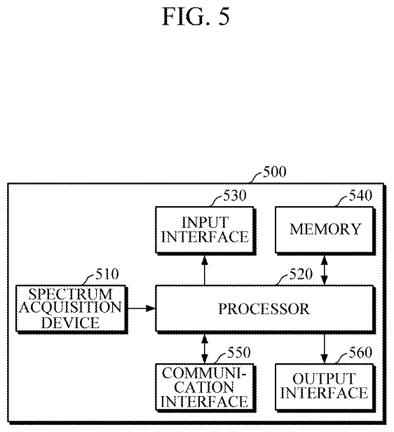

FIG. 5 is a block diagram illustrating another example of an apparatus for estimating a concentration. The concentration estimating apparatus of FIG. 5 is an apparatus for estimating an analyte concentration by analyzing an in vivo spectrum of an object, and may be mounted in various electronic devices described above or may be enclosed in a housing to be provided as a separate device.

Referring to FIG. 5, the concentration estimating apparatus 500 includes a spectrum acquisition device 510, a processor 520, an input interface 530, a memory 540, a communication interface 550, and an output interface 560. Here, the spectrum acquisition device 510 and the processor 520 may be substantially similar as the spectrum acquisition device 310 and the processor 320 described above with reference to FIG. 3, such that detailed description thereof may be omitted.

The input interface 530 may receive input of various operation signals based on a user input. In an embodiment, the input part 530 may include a keypad, a dome switch, a touch pad (e.g., a static pressure touch pad, a capacitive touch page, or the like), a jog wheel, a jog switch, a hardware (H/W) button, and the like. Particularly, the touch pad, which forms a layer structure with a display, may be referred to as a touch screen.

The memory 540 may store programs or commands for operation of the concentration estimating apparatus 500, and may store data input to and output from the concentration estimating apparatus 500. Further, the memory 540 may store an in vivo spectrum, a concentration estimation model, an estimated analyte concentration value, and the like. The memory 540 may include at least one storage medium of a flash memory type memory, a hard disk type memory, a multimedia card micro type memory, a card type memory (e.g., a secure digital (SD) memory, an eXtreme digital (XD) memory, etc.), a Random Access Memory (RAM), a Static Random Access Memory (SRAM), a Read Only Memory (ROM), an Electrically Erasable Programmable Read Only Memory (EEPROM), a Programmable Read Only Memory (PROM), a magnetic memory, a magnetic disk, an optical disk, and the like. Further, the concentration estimating apparatus 500 may communicate with an external storage medium, such as web storage and the like, which performs a storage function of the memory 540 via the Internet.

The communication interface 550 may perform communication with an external device. For example, the communication interface 550 may transmit, to the external device, the data input to the concentration estimating apparatus 500, data stored in and processed by the concentration estimating apparatus 500, and the like, or may receive, from the external device, various data for generating a concentration estimation model and estimating an analyte concentration.

In this case, the external device may be medical equipment using the data input to the concentration estimating apparatus 500, the data stored in and processed by the concentration estimating apparatus 500, and the like, a printer to print out results, or a display to display the results. In addition, the external device may be a digital television (TV), a desktop computer, a cellular phone, a smartphone, a tablet PC, a laptop computer, a personal digital assistant (PDA), a portable multimedia player (PMP), a navigation device, an MP3 player, a digital camera, a wearable device, and the like, but is not limited thereto.

The communication interface 550 may communicate with an external device by using Bluetooth communication, Bluetooth Low Energy (BLE) communication, Near Field Communication (NFC), WLAN communication, Zigbee communication, Infrared Data Association (IrDA) communication, Wi-Fi Direct (WFD) communication, Ultra-Wideband (UWB) communication, Ant+ communication, Wi-Fi communication, Radio Frequency Identification (RFID) communication, 3G communication, 4G communication, 5G communication, and the like. However, this is merely exemplary and is not intended to be limiting.

The output interface 560 may output the data input to the concentration estimating apparatus 500, the data stored in and processed by the concentration estimating apparatus 500, and the like. In one embodiment, the output interface 560 may output the data input to the concentration estimating apparatus 500, the data stored in and processed by the concentration estimating apparatus 500, and the like, by using at least one of an acoustic method, a visual method, and a tactile method. To this end, the output part 560 may include a speaker, a display, a vibrator, and the like.

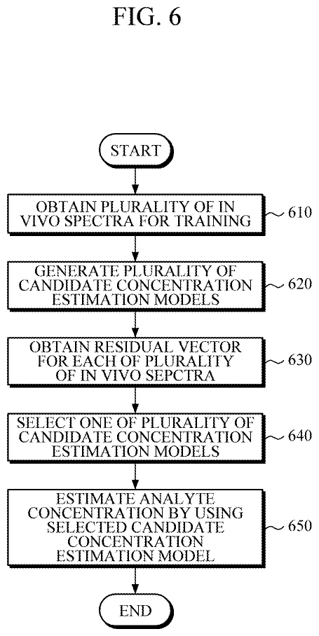

FIG. 6 is a flowchart illustrating an example of a method of estimating a concentration. The concentration estimating method of FIG. 6 may be performed by the concentration estimating apparatuses 100 and 500 of FIG. 1 or 5, respectively.

Referring to FIG. 6, the concentration estimating apparatus may obtain a plurality of in vivo spectra for training, which are measured during an interval in which an analyte concentration is substantially constant, in operation 610. For example, the concentration estimating apparatus may obtain a plurality of in vivo spectra for training by receiving the in vivo spectra from an external device which measures and/or store in vivo spectra. Alternatively, the concentration estimating apparatus may obtain the in vivo spectra by directly measuring the in vivo spectra by emitting light towards an object and receiving light reflected by or scattered from the object during an interval (e.g., timeframe) in which an analyte concentration of an object is substantially constant.

Based on obtaining the plurality of in vivo spectra for training, the concentration estimating apparatus may generate a plurality of candidate concentration estimation models by varying the number of principal components based on the plurality of obtained in vivo spectra for training in operation 620. In an embodiment, the concentration estimating apparatus may generate the plurality of candidate concentration estimation models based on the NAS algorithm by using the plurality of in vivo spectra for training. In this case, examples of the analyte may include glucose, triglycerides, urea, uric acid, lactate, proteins, cholesterol, ethanol, and the like, but the analyte is not limited thereto. In the case where an in vivo analyte is glucose, an analyte concentration may indicate a blood glucose level; and an interval in which an analyte is substantially constant may indicate a fasting interval in which glucose is not consumed by an object. For example, the concentration estimating apparatus may extract a predetermined number of principal component vectors by analyzing the plurality of in vivo spectra for training using various dimension reduction algorithms such as Principal Component Analysis (PCA). Independent Component Analysis (ICA), Non-negative Matrix Factorization (NMF), Singular Value Decomposition (SVD), and the like. Further, upon varying the number of principal components, the concentration estimating apparatus may obtain an inverse matrix of a matrix composed of the varied number of principal component vectors and the pure component spectrum vector of an analyte, to generate a plurality of candidate concentration estimation model matrices; and may generate a plurality of candidate concentration estimation models based on the plurality of generated candidate concentration estimation model matrices.

The concentration estimating apparatus may obtain a residual vector for each of the plurality of in vivo spectra for training, by using the plurality of candidate concentration estimation models generated by varying the number of principal components in operation 630. In this case, the residual vector may represent a difference between an in vivo spectrum, reconstructed using the concentration estimation model, and an actually measured in vivo spectrum. For example, the concentration estimating apparatus may determine principal component concentrations C_{n,1}, C_{n,2}, and C_{n,k}, and an analyte concentration C_{n,g} for each of the in vivo spectra for training by using Equation 1 as described elsewhere herein, and may reconstruct each of the in vivo spectra for training by using Equation 2 as described elsewhere herein, to generate a vector Sᵣₑ_{_{n,k}} of the reconstructed in vivo spectrum for training. In addition, the concentration estimating apparatus may obtain a residual vector Residual_{n,k} of each of the in vivo spectra for training by using Equation 3 as described elsewhere herein.

The concentration estimating apparatus may select one of the plurality of candidate concentration estimation models generated by varying the number of principal components based on the residual vector in operation 640. For example, the concentration estimating apparatus may extract a component vector glucoseₖ_NAS, corresponding to an analyte, from the plurality of generated candidate concentration estimation model matrices generated by varying the number of principal components; and may determine an angle θ_{n,k} between the component vector glucoseₖ_NAS corresponding to the analyte and the residual vector Residual_{n,k}, and may determine the number k of principal components, at which a value obtained by multiplying a magnitude of the residual vector Residual_{n,k} by an absolute value of cos θ_{n,k} is maximum. Further, among the plurality of candidate concentration estimation models generated for each number of principal components, the concentration estimating apparatus may select a candidate concentration estimation model generated by using the determined number k of principal components.

The concentration estimating apparatus may estimate an analyte concentration by using the selected candidate concentration estimation model in operation 650. For example, the concentration estimating apparatus may obtain an in vivo spectrum for estimation, which is measured for estimating an analyte concentration of an object, and may estimate the analyte concentration of the object by using the obtained in vivo spectrum for estimation and the selected candidate concentration estimation model.

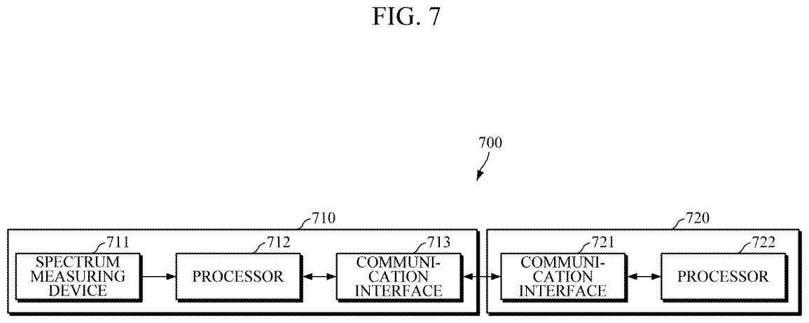

FIG. 7 is a block diagram illustrating an example of a system for estimating a concentration. The concentration estimating system 700 of FIG. 7 may be an example of a system in which the function of generating a concentration estimation model and the function of estimating a concentration, which are described above with reference to FIGS. 3 through 6, are performed by separate apparatuses. The function of estimating a concentration may be performed by a concentration estimating apparatus 710, and the function of generating a concentration estimation model may be performed by a model generating apparatus 720.

More specifically, by using the spectrum measuring device 711, the concentration estimating apparatus 710 may measure an in vivo spectrum for training by emitting light towards an object and receiving light reflected by or scattered from the object during an interval in which an analyte concentration of an object is substantially constant; and may transmit the measured in vivo spectrum for training to the model generating apparatus 720 via a communication interface 713.

The model generating apparatus 720 may receive the in vivo spectrum for training from the concentration estimating apparatus 710 via the communication interface 721; may generate a plurality of candidate concentration estimation models by using the in vivo spectrum for training via the processor 722; and may select one of the plurality of candidate concentration estimation models as a concentration estimation model. Further, the model generating apparatus 720 may transmit the selected concentration estimation model to the concentration estimating apparatus 710 via the communication interface 721.

The concentration estimating apparatus 710 may receive the concentration estimation model from the model generating apparatus 720 via the communicator 713, and may measure an in vivo spectrum for estimation by emitting light towards an object and receiving light reflected by or scattered from the object via the spectrum measuring device 711. In addition, the concentration estimating apparatus 710 may estimate an analyte concentration using the in vivo spectrum for estimation and the concentration estimation model via the processor 712.

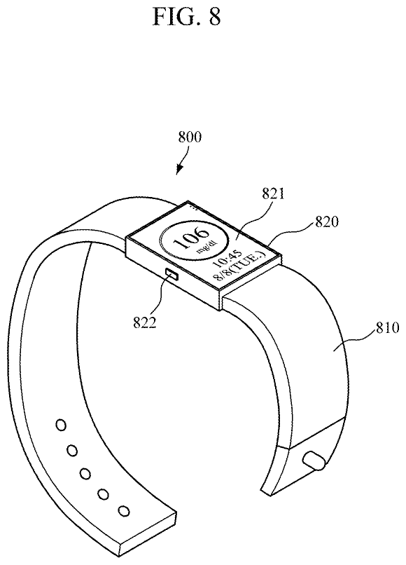

FIG. 8 is a diagram illustrating an example of a wrist-type wearable device.

Referring to FIG. 8, the wrist-type wearable device 800 includes a strap 810 and a main body 820.

The strap 810 may be connected to both ends of the main body 820 in a detachable manner, or may be integrally formed therewith as a smart band. The strap 810 may be made of a flexible material so as to conform to a user's wrist.

The main body 820 may include the concentration estimating apparatuses 300, 500, and 710 described above. Further, the main body 820 may include a battery which supplies power to the wrist-type wearable device 800 and the concentration estimating apparatuses 300, 500, and 710.

An optical sensor may be disposed on the bottom of the main body 820 to be exposed toward a user's wrist. Accordingly, when a user wears the wrist-type wearable device 800, the optical sensor may naturally come into contact with the user's skin. In this case, the optical sensor may obtain an in vivo spectrum by emitting light towards an object and receiving light reflected or scattered from the object.

The wrist-type wearable device 800 may further include a display 821 and an input interface 822 which are mounted at the main body 820. The display 821 may display data processed by the wrist-type wearable device 800 and the concentration estimating apparatuses 300, 500, and 710, processing results data thereof, and the like. The input interface 822 may receive various operation signals from a user.

The present disclosure may be realized as a computer-readable code stored in a non-transitory computer-readable medium. The computer-readable medium may be any type of recording medium in which data is stored in a computer-readable manner. Examples of the computer-readable medium include a ROM, a RAM, a CD-ROM, a magnetic tape, a floppy disc, an optical data storage, and a carrier wave (e.g., data transmission through the Internet). The computer-readable medium may be distributed over a plurality of computer systems connected via a network so that a computer-readable code is written thereto and executed therefrom in a decentralized manner. Functional programs, code, and code segments for implementing the embodiments of the present disclosure may be easily deduced by one of ordinary skill in the art.

The present disclosure has been described herein with regard to the embodiments. However, it should be apparent to those skilled in the art that various changes and modifications may be made without deviating from the technical concepts and features of the present disclosure. Thus, it is clear that the above-described embodiments are illustrative in all aspects and are not intended to limit the present disclosure.

What is claimed is:

1. An apparatus for estimating an analyte concentration, the apparatus comprising:
a spectrum acquisition device configured to obtain a plurality of in vivo spectra for training which are measured during a first interval, and obtain an in vivo spectrum for analyte concentration estimation which is measured during a second interval; and
a processor configured to:
generate a plurality of candidate concentration estimation models by varying a number of principal components based on the plurality of in vivo spectra for training;
obtain a plurality of residual vectors corresponding to the plurality of in vivo spectra for training by using the plurality of candidate concentration estimation models;
select a candidate concentration estimation model, from among the plurality of candidate concentration estimation models, based on the plurality of residual vectors; and
estimate the analyte concentration by using the selected candidate concentration estimation model and the in vivo spectrum for analyte concentration estimation.

2. The apparatus of claim 1, wherein the processor is configured to generate the plurality of candidate concentration estimation models using a Net Analyte Signal (NAS) algorithm.

3. The apparatus of claim 1, wherein the plurality of residual vectors represent differences between generated in vivo spectra, generated using the plurality of concentration estimation models, and actually measured in vivo spectra.

4. The apparatus of claim 1, wherein the processor is further configured to:
extract a predetermined number of principal component vectors by analyzing the plurality of in vivo spectra for training;
based on varying the number of principal components, obtain a plurality of inverse matrices of matrices composed of the varied number of principal component vectors and a pure component spectrum vector of an analyte;
generate a plurality of candidate concentration estimation model matrices based on the plurality of inverse matrices; and
generate the plurality of candidate concentration estimation models based on the plurality of candidate concentration estimation model matrices.

5. The apparatus of claim 4, wherein the processor is configured to extract the predetermined number of principal component vectors by using one of Principal Component Analysis (PCA), Independent Component Analysis (ICA), Non-negative Matrix Factorization (NMF), and Singular Value Decomposition (SVD).

6. The apparatus of claim 4, wherein the processor is configured to:
extract a plurality of component vectors, corresponding to the analyte, from the plurality of candidate concentration estimation model matrices;
determine angles between the plurality of extracted component vectors and the plurality of residual vectors;
determine a number of principal components, at which a value obtained by multiplying a magnitude of a residual vector, of the plurality of residual vectors, by an absolute value of cosine of the angle is maximum; and
select the candidate concentration estimation model, generated by using the determined number of principal components, from among the plurality of generated candidate concentration estimation models.

7. The apparatus of claim 1, wherein the spectrum acquisition device is configured to receive the plurality of in vivo spectra for training and the in vivo spectrum for analyte concentration estimation from an external device.

8. The apparatus of claim 1, wherein the spectrum acquisition device is configured to measure the plurality of in vivo spectra for training and the in vivo spectrum for analyte concentration estimation by emitting light towards an object and receiving light reflected by or scattered from the object.

9. The apparatus of claim 1, wherein the first interval is an interval in which the analyte concentration of is substantially constant.

10. The apparatus of claim 1, wherein the analyte is at least one of glucose, triglycerides, urea, uric acid, lactate, proteins, cholesterol, or ethanol.

11. The apparatus of claim 1, wherein:
the analyte is glucose; and
the first interval is a fasting interval.

12. A method of estimating an analyte concentration, the method comprising:
obtaining a plurality of in vivo spectra for training which are measured during a predetermined interval;
generating a plurality of candidate concentration estimation models by varying a number of principal components based on the plurality of in vivo spectra for training;
obtaining a plurality of residual vectors corresponding to the plurality of in vivo spectra for training by using the plurality of candidate concentration estimation models;
selecting a candidate concentration estimation model, from among the plurality of candidate concentration estimation models, based on the plurality of residual vectors; and
estimating the analyte concentration by using the selected concentration estimation model.

13. The method of claim 12, wherein the generating of the plurality of candidate concentration estimation models by varying the number of principal components comprises generating the plurality of candidate concentration estimation models using a Net Analyte Signal (NAS) algorithm.

14. The method of claim 12, wherein the plurality of residual vectors represent differences between a plurality of generated in vivo spectrum, generating using the plurality of concentration estimation models, and a plurality of actually measured in vivo spectra.

15. The method of claim 12, wherein the generating of the plurality of candidate concentration estimation models by varying the number of principal components comprises:
extracting a predetermined number of principal component vectors by analyzing the plurality of in vivo spectra for training;
based on varying the number of principal components, obtaining a plurality of inverse matrices of matrices composed of the varied number of principal component vectors and a pure component spectrum vector of an analyte;
generating a plurality of candidate concentration estimation model matrices based on the plurality of inverse matrices; and
generating the plurality of candidate concentration estimation models based on the plurality of candidate concentration estimation model matrices.

16. The method of claim 15, wherein the extracting of the predetermined number of principal component vectors comprises extracting the predetermined number of principal component vectors by using one of Principal Component Analysis (PCA), Independent Component Analysis (ICA), Non-negative Matrix Factorization (NMF), and Singular Value Decomposition (SVD).

17. The method of claim 15, wherein the selecting of the candidate concentration estimation model comprises:
extracting a plurality of component vectors, corresponding to the analyte, from the plurality of candidate concentration estimation model matrices;
determining angles between the plurality of component vectors and the plurality of residual vectors;
determining a number of principal components, at which a value obtained by multiplying a magnitude of a residual vector, of the plurality of residual vectors, by an absolute value of cosine of the angle is maximum; and
selecting the candidate concentration estimation model, generated by using the determined number of principal components, from among the plurality of candidate concentration estimation models.

18. The method of claim 12, wherein the predetermined interval is an interval in which the analyte concentration is substantially constant.

19. The method of claim 12, wherein the analyte is at least one of glucose, triglycerides, urea, uric acid, lactate, proteins, cholesterol, or ethanol.

20. The method of claim 12, wherein:
the analyte is glucose; and
the predetermined interval is a fasting interval.
